(19) United States
(12) Patent Application Publication
(10) Pub. No.: US 2024/0269127 A1
(43) Pub. Date: Aug. 15, 2024

(54) QUINOLINE COMPOUND SUSTAINED-RELEASE TABLET AND PREPARATION METHOD THEREFOR

(71) Applicant: Hinova Pharmaceuticals Inc., Chengdu (CN)

(72) Inventors: Xing Wei, Chengdu (CN); Tongtao Kuang, Chengdu (CN); Jiang Chen, Chengdu (CN); Chaowu Ai, Chengdu (CN); Xinghai Li, Chengdu (CN)

(73) Assignee: Hinova Pharmaceuticals Inc., Chengdu (CN)

(21) Appl. No.: 18/608,261

(22) Filed: Mar. 18, 2024

Related U.S. Application Data
(63) Continuation-in-part of application No. PCT/CN2021/119132, filed on Sep. 15, 2021.

(51) Int. Cl. A61K 31/47; A61K 9/20; A61K 9/28; C07D 215/36

(52) U.S. Cl. CPC A61K 31/47; A61K 9/2013; A61K 9/2018; A61K 9/2054; A61K 9/2095; A61K 9/2866; C07D 215/36

(57) ABSTRACT

This patent document provides a sustained-release formulation comprising 1-40 parts of a quinoline compound, 100-300 parts of a filler, 50-200 parts of a sustained-release material and optionally 0.5-4 parts of a lubricant. Also provided are methods of treating diseases with the sustained-release formulation.

### CROSS-REFERENCE TO RELATED APPLICATIONS

This application is a continuation-in-part of International Patent Application No. PCT/CN2021/119132, filed on Sep. 17, 2021, the disclosure of which is incorporated herein by reference in its entirety.

### TECHNOLOGY OF THE INVENTION

The present invention specifically relates to a quinoline compound sustained-release tablet and a preparation method therefor, and belongs to the pharmaceutical field.

### BACKGROUND OF THE INVENTION

Quinoline compounds are the inhibitors of urate anion transporter 1 (URAT1), which are self-developed by Hinova Pharmaceuticals Inc., possessing independent intellectual property rights (currently, 8 invention patents have been applied), and used for the treatment of hyperuricemia and gout. The active ingredients of quinoline compound sustained-release tablets are quinoline compounds, having a structure represented by formula I:

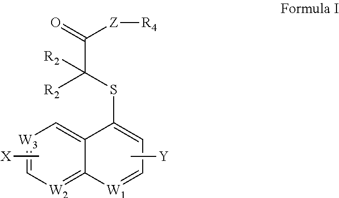

Application number: CN201611109936.3, invention name: Quinoline compounds, preparation method thereof, and use thereof as urate transporter inhibitor drug.

According to the PK test results of the drug in rats (FIG. 1), the drug is quickly absorbed after entering the rat body, and the rats have a high plasma concentration for 4 hours after receiving the drug. Based on the pharmacological experiments, it is concluded that the excessive plasma concentration can cause potential toxic side effects. The CACO-2 experiment has shown that quinoline compounds exhibit high permeability in CACO-2 cells and are not substrates for efflux transporters. The solubility test has indicated that quinoline compounds have low solubility in water, and so they are classified as BCS class 2 according to biopharmaceutics classification system. Drug release is the rate-limiting process for drug absorption. Generally, there is a high correlation between the in vitro drug release testing and the in vivo drug bioavailability. Therefore, the development of sustained-release formulations can effectively reduce the plasma concentration after medication and the potential toxic side effects caused by Cₘₐₓ in the human body, while ensure the bioavailability.

### SUMMARY

The object of the present invention is to provide a quinoline compound sustained-release tablet and a preparation method therefor. In the present invention, the concept of sustained-release formulations is used in the development of the compound preparations, with the development goal of administrating once a day, effectively reducing Cₘₐₓ while maintaining AUC unchanged. The determined formulation is fully released within 12-20 h, improves drug compliance, reduces the incidence of adverse reactions, and increases drug stability.

The present invention provides a quinoline compound sustained-release tablet, which contains the following raw and auxiliary materials in parts by weight:
1-40 parts of quinoline compounds, 100-300 parts of fillers, 50-200 parts of sustained-release materials and 0.5-4 parts of lubricants;
the filler comprises, but is not limited to, mannitol, microcrystalline cellulose, lactose, starch, corn starch, calcium hydrogen phosphate hydrate, magnesium carbonate, calcium carbonate, purified sucrose and/or glucose;
the sustained-release material comprises, but is not limited to, hydroxypropyl methylcellulose, hydroxypropyl cellulose, povidones, glyceryl behenate, and/or long-chain fatty acids;
the lubricant comprises, but is not limited to, magnesium stearate, calcium stearate, sucrose fatty acid ester, sodium stearyl fumarate, polyethylene glycol, talc, glyceryl behenate and/or stearic acid.

Further, the filler is lactose and/or microcrystalline cellulose, and preferably, the filler is composed of lactose and microcrystalline cellulose at a mass ratio of 10-30:2-10; the sustained-release material is hydroxypropyl methylcellulose; the lubricant includes but is not limited to magnesium stearate, sodium fumarate, glyceryl behenate, and/or stearic acid.

More further, said lactose includes but is not limited to lactose monohydrate, anhydrous lactose, and/or lactose with different particle sizes; said microcrystalline cellulose includes but is not limited to microcrystalline cellulose PH101, PH102, PH301, and/or PH302; said hydroxypropyl methylcellulose includes but is not limited to hydroxypropyl methylcellulose K4M, K100LV, and/or K750; and said lubricant is magnesium stearate.

More further, the sustained-release tablet comprises the following raw and auxiliary materials in parts by weight:
5 parts of quinoline compounds, 256 parts of fillers, 117 parts of sustained-release materials and 1.9 parts of lubricants;
the filler is composed of lactose monohydrate and microcrystalline cellulose PH101 at a mass ratio of 10-70:2-30;
said hydroxypropyl methylcellulose is composed of hydroxypropyl methylcellulose K4M and K100LV, and their mass ratio is preferably 57:60;
and said lubricant is magnesium stearate.

Further, the quinoline compound is a compound as represented by formula I, or an optical isomer thereof, or a solvate thereof, or a pharmaceutically acceptable salt thereof, or a prodrug thereof;

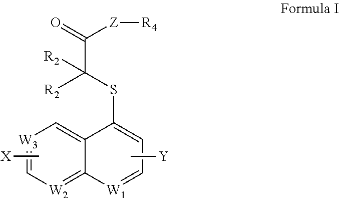

wherein:
Z is selected from O, S or —NH—;
W₁ is selected from N or CR^{a}; W₂ is selected from N or CR^{b}; W₃ is selected from N or CR^{c};
R^{a}, R^{b}, R^{c}, R₂, and R₃ are each independently selected from the group consisting of hydrogen, halogen, cyano, nitro, alkyl, alkenyl, alkynyl, cycloalkyl, heterocyclyl, aryl, heteroaryl, —OR^{d}, —S(O)ₘR^{d}, —C(O)R^{d}, C(O)OR^{d}, —C(O)NR^{e}R^{f}, —NR^{e}R^{f} or NR^{e}C(O)R^{f}, in which said alkyl, alkenyl, alkynyl, cycloalkyl, heterocyclyl, aryl or heteroaryl are each independently and optionally further substituted with one or more of the substituents selected from the group consisting of halogen, cyano, nitro, oxo, alkyl, haloalkyl, hydroxyalkyl, alkenyl, alkynyl, cycloalkyl, heterocyclyl, aryl, heteroaryl, —OR^{d}, —S(O)ₘR^{d}, —C(O)R^{d}, C(O)OR^{d}, —C(O)NR^{e}R^{f}, —NR^{e}R^{f} or NR^{e}C(O)R^{f};
R^{d} is selected from the group consisting of hydrogen, halogen, alkyl, cycloalkyl, heterocyclyl, aryl or heteroaryl, wherein said alkyl, cycloalkyl, heterocyclyl, aryl or heteroaryl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, carboxylic ester group, —C(O)NR^{e}R^{f}, —NR^{e}R^{f} or NR^{e}C(O)R^{f};
R^{e} and R^{f} are each independently selected from the group consisting of hydrogen, alkyl, cycloalkyl, heterocyclyl, aryl or heteroaryl, wherein said alkyl, cycloalkyl, heterocyclyl, aryl or heteroaryl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, and carboxylic ester group; and m is 0, 1, or 2;
X and Y are each independently selected from the group consisting of hydrogen, halogen, cyano, nitro, alkyl, cycloalkyl, haloalkyl and hydroxyalkyl;
Provided that Z is selected from O or S, R₄ is selected from hydrogen or C₁-C₆ alkyl, cycloalkyl, wherein said alkyl and cycloalkyl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, and carboxylic ester group, —C(O)NR^{e}R^{f}, —NR^{e}R^{f} or NR^{e}C(O)R^{f}; provided that Z is —NH—, R₄ is selected from the group consisting of hydrogen, aryl or heteroaryl, and preferably pyridinyl.

More further, the quinoline compound is 2-[4-(6-bromoquinolyl)-thio]-2-ethylbutyric acid and/or an optical isomer, a solvate, a pharmaceutically acceptable salt, and a prodrug thereof; the structure of said 2-[4-(6-bromoquinolyl)-thio]-2-ethylbutyric acid is as follows:

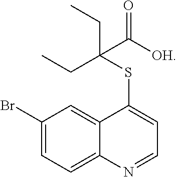 [SMILES: CCC(CC)(Sc1ccnc2ccc(Br)cc12)C(=O)O]

More further, the quinoline compound is crystalline form I, crystalline form II and/or a sodium salt crystalline form of 2-[4-(6-bromoquinolyl)-thio]-2-ethylbutyric acid. The X-ray powder diffraction pattern is generally obtained when the compound or its salt is irradiated with a Cu-Kα light source.

More further, there are characteristic peaks where diffraction angles 2θ are 6.5±0.2°, 13.6±0.2°, 14.1±0.2°, 17.7±0.2°, 21.8±0.2°, 22.0±0.2°, 22.8±0.2°, 23.2±0.2°, 24.3±0.2°, 26.8±0.2°, and 27.4±0.2° in the X-ray powder diffraction pattern of the crystalline form I of 2-[4-(6-bromoquinolyl)thio]-2-ethylbutyric acid.

More further, the relative intensity of the characteristic peak is:

| Diffraction angle 2θ | Relative intensity % |
|---|---|
| 6.5 ± 0.2 | 36.1 |
| 13.6 ± 0.2 | 100.0 |
| 14.1 ± 0.2 | 14.1 |
| 17.7 ± 0.2 | 13.6 |
| 21.8 ± 0.2 | 17.0 |
| 22.0 ± 0.2 | 11.1 |
| 22.8 ± 0.2 | 16.9 |
| 23.2 ± 0.2 | 11.1 |
| 24.3 ± 0.2 | 45.6 |
| 26.8 ± 0.2 | 15.7 |
| 27.4 ± 0.2 | 36.5. |

More further, there are characteristic peaks where diffraction angles 2θ are 7.9±0.2°, 9.5±0.2°, 15.9±0.2°, 18.2±0.2°, 19.1±0.2°, 23.9±0.2°, and 26.1±0.2° in the X-ray powder diffraction pattern of the crystalline form II of 2-[4-(6-bromoquinolyl)thio]-2-ethylbutyric acid.

More further, the relative intensity of the characteristic peak is:

| Diffraction angle 2θ | Relative intensity % |
|---|---|
| 7.9 ± 0.2 | 100.0 |
| 9.5 ± 0.2 | 7.3 |
| 15.9 ± 0.2 | 28.6 |
| 18.2 ± 0.2 | 10.0 |
| 19.1 ± 0.2 | 6.8 |
| 23.9 ± 0.2 | 22.7 |
| 26.1 ± 0.2 | 6.7. |

More further, there are characteristic peaks where diffraction angles 2θ are 6.1±0.2°, 10.5±0.2°, 12.0±0.2°, 14.1±0.2°, 15.9±0.2°, 18.0±0.2°, 21.7±0.2°, 27.6±0.2°, 32.0±0.2°, 33.8±0.2°, and 36.4±0.2° in the X-ray powder diffraction pattern of the sodium salt crystalline form of 2-[4-(6-bromoquinolyl)thio]-2-ethylbutyric acid.

More further, the relative intensity of the characteristic peak is:

| Diffraction angle 2θ | Relative intensity % |
|---|---|
| 6.1 ± 0.2 | 22.2 |
| 10.5 ± 0.2 | 21.4 |
| 12.0 ± 0.2 | 30.7 |
| 14.1 ± 0.2 | 24.3 |
| 15.9 ± 0.2 | 89.9 |
| 18.0 ± 0.2 | 38.5 |
| 21.7 ± 0.2 | 27.9 |
| 27.6 ± 0.2 | 100.0 |
| 32.0 ± 0.2 | 22.0 |
| 33.8 ± 0.2 | 34.2 |
| 36.4 ± 0.2 | 22.4. |

The present invention also provides a method for preparation of the sustained-release tablet mentioned above, and the preparation of crystalline form I of 2-[4-(6-bromoquinolyl)-thio]-2-ethylbutyric acid comprises the following steps:
the crude product of 2-[4-(6-bromoquinolyl)-thio]-2-ethylbutyric acid was dissolved in methanol, and then filtered. The filtrate was naturally cooled and allowed to crystallize for 20-24 h. Then, the temperature was controlled to be 15-20° C., and the filtrate was stirred and allowed to crystallized for 2 h, followed by filtration. The crystals were dried to obtain crystalline form I.

The present invention also provides a method for preparation of the sustained-release tablet mentioned above, and the preparation of crystalline form II of 2-[4-(6-bromoquinolyl)-thio]-2-ethylbutyric acid comprises the following steps:
the crude product of 2-[4-(6-bromoquinolyl)-thio]-2-ethylbutyric acid was dissolved in a mixed solution of tetrahydrofuran and dichloromethane (7:4, v/v), and then filtered. The filtrate was evaporated to dry at room temperature to obtain crystalline form II.

The present invention also provides a method for preparation of the sustained-release tablet mentioned above, and the preparation of said sodium salt crystalline form of 2-[4-(6-bromoquinolyl)-thio]-2-ethylbutyric acid comprises the following steps:
the crude product of 2-[4-(6-bromoquinolyl)-thio]-2-ethylbutyric acid was dissolved in water, and the pH value was adjusted to be 12 with NaOH aqueous solution. The resultant solution was stirred for 3 min and filtered. The crystal was dried in vacuum to obtain the sodium salt crystalline form.

The present invention also provides a method for preparation of the quinoline compound sustained-release tablet mentioned above, which is prepared using wet granulation, dry granulation, direct powder compression or membrane controlled-release techniques, and preferably wet granulation.

Further, the wet granulation comprises the following steps:
a) The raw and adjuvant materials are weighed according to the ratio in the formula, and lactose is passed through a 60-mesh sieve;
b) ¼-⅓ of the pre-determined amount of lactose in the formula is placed in a wet granulator, stirred for 5-10 min, and then ¼-⅓ of the pre-determined amount of lactose and ⅓-½ of the pre-determined amount of quinoline compound are added and stirred for 5-10 min. Finally, the remaining amounts of lactose and quinoline compounds are added, and stirred for 5-10 min. Then, microcrystalline cellulose and hydroxypropyl methylcellulose are added and stirred for 10-20 min;
c) Water is added to make soft materials and granules, and then wet granules are dried to a moisture content of 1.0%˜4.0%, followed by sorting;
d) Magnesium stearate is mixed with the granules prepared in step c, and then the mixture is pressed and coated to obtain the tablets.

More further, in step b, ⅓ of the pre-determined amount of lactose in the formula is placed in a wet granulator, stirred for 5 min, and then ⅓ of the pre-determined amount of lactose and ½ of the pre-determined amount of quinoline compound are added and stirred for 5 min. Finally, the remaining amounts of lactose and quinoline compounds are added, and stirred for 5 min. Then, microcrystalline cellulose and hydroxypropyl methylcellulose are added and stirred for 10 min.

More further, in step c, the granulation process involves passing through a 20-mesh sieve, the drying is carried out at 60° C., and the sorting is performed with a 20-mesh sieve; and/or, in step d, the tablet is coated at a weight gain of 2-4%.

The present invention also provides a crystalline form of 2-[4-(6-bromoquinolyl)-thio]-2-ethylbutyric acid, which is crystalline form I of 2-[4-(6-bromoquinolyl)-thio]-2-ethylbutyric acid, and there are characteristic peaks where diffraction angles 2θ are 6.5±0.2°, 13.6±0.2°, 14.1±0.2°, 17.7±0.2°, 21.8±0.2°, 22.0-0.2°, 22.8-0.2°, 23.2=0.2°, 24.3=0.2°, 26.8=0.2°, and 27.4±0.2° in the X-ray powder diffraction pattern thereof.

Further, the relative intensity of the characteristic peaks is:

| Diffraction angle 2θ | Relative intensity % |
|---|---|
| 6.5 ± 0.2 | 36.1 |
| 13.6 ± 0.2 | 100.0 |
| 14.1 ± 0.2 | 14.1 |
| 17.7 ± 0.2 | 13.6 |
| 21.8 ± 0.2 | 17.0 |
| 22.0 ± 0.2 | 11.1 |
| 22.8 ± 0.2 | 16.9 |
| 23.2 ± 0.2 | 11.1 |
| 24.3 ± 0.2 | 45.6 |
| 26.8 ± 0.2 | 15.7 |
| 27.4 ± 0.2 | 36.5. |

The present invention also provides a crystalline form of 2-[4-(6-bromoquinolyl)-thio]-2-ethylbutyric acid, which is crystalline form II of 2-[4-(6-bromoquinolyl)-thio]-2-ethylbutyric acid, and there are characteristic peaks where diffraction angles 2θ are 7.9±0.2°, 9.5±0.2°, 15.9±0.2°, 18.2±0.2°, 19.1±0.2°, 23.9±0.2°, and 26.1±0.2° in the X-ray powder diffraction pattern thereof.

Further, the relative intensity of the characteristic peaks is:

| Diffraction angle 2θ | Relative intensity % |
|---|---|
| 7.9 ± 0.2 | 100.0 |
| 9.5 ± 0.2 | 7.3 |
| 15.9 ± 0.2 | 28.6 |
| 18.2 ± 0.2 | 10.0 |
| 19.1 ± 0.2 | 6.8 |
| 23.9 ± 0.2 | 22.7 |
| 26.1 ± 0.2 | 6.7. |

The present invention also provides a crystalline form of 2-[4-(6-bromoquinolyl)-thio]-2-ethylbutyric acid, which is the sodium salt crystalline form of 2-[4-(6-bromoquinolyl)-thio]-2-ethylbutyric acid, and there are characteristic peaks where diffraction angles are 6.1±0.2°, 10.5±0.2°, 12.0±0.2°, 14.1±0.2°, 15.9±0.2°, 18.0±0.2°, 21.7±0.2°, 27.6±0.2°, 32.0±0.2°, 33.8±0.2°, and 36.4±0.2° in the X-ray powder diffraction pattern thereof.

Further, the relative intensity of the characteristic peaks is:

| Diffraction angle 2θ | Relative intensity % |
|---|---|
| 6.1 ± 0.2 | 22.2 |
| 10.5 ± 0.2 | 21.4 |
| 12.0 ± 0.2 | 30.7 |
| 14.1 ± 0.2 | 24.3 |
| 15.9 ± 0.2 | 89.9 |
| 18.0 ± 0.2 | 38.5 |
| 21.7 ± 0.2 | 27.9 |
| 27.6 ± 0.2 | 100.0 |
| 32.0 ± 0.2 | 22.0 |
| 33.8 ± 0.2 | 34.2 |
| 36.4 ± 0.2 | 22.4 |

Another aspect provides a pharmaceutical composition comprising a therapeutically effective amount of the aforementioned crystalline Form 1, crystalline Form 1, or their respective pharmaceutically acceptable salt thereof. In some embodiments, the pharmaceutical composition comprises a therapeutically effective amount of the sodium salt crystalline form of 2-[4-(6-bromoquinolyl)-thio]-2-ethylbutyric acid as defined above.

The present invention has the following advantages:
1. Under the conditions of ensuring that the drug bioavailability is not decreased, the highest plasma concentration is reduced, avoiding potential toxic side effects;
2. Administrating once a day greatly improves patient compliance;
3. The selected excipients are all within the safe dosage specified by FDA and SFDA, and have good compatibility with the active ingredients, resulting in high product stability;
4. The sustained-release tablets of quinoline compounds are prepared using common solid preparation techniques, which have high production efficiency and low costs.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. The in vivo drug time curve of active ingredient (formerly code-named HC-1310) in rats

FIG. 2. XRD pattern of crystalline form I.

FIG. 3. DSC spectroscopy of crystalline form I.

FIG. 4. TGA spectrum of crystalline form I.

FIG. 5. PLM pattern of crystalline form I.

FIG. 6. XRD pattern of crystalline form II.

FIG. 7. DSC spectroscopy of crystalline form II.

FIG. 8. TGA spectrum of crystalline form II.

FIG. 9. PLM pattern of crystalline form II.

FIG. 10. XRD pattern of sodium salt crystalline form.

FIG. 11. DSC spectroscopy of sodium salt crystalline form.

FIG. 12. TGA spectrum of sodium salt crystalline form.

FIG. 13. Screening of sustained-release material types and release curves of samples at different ratios.

FIG. 14. Comparison of release curves of quinoline compound sustained-release tablets (specification: 5 mg) coated and not coated (plain tablets).

FIG. 15. The mean plasma concentration-time curve of quinoline compound suspension or sustained-release tablets in male and female beagle dogs after a single oral administration at 5 mg/kg.

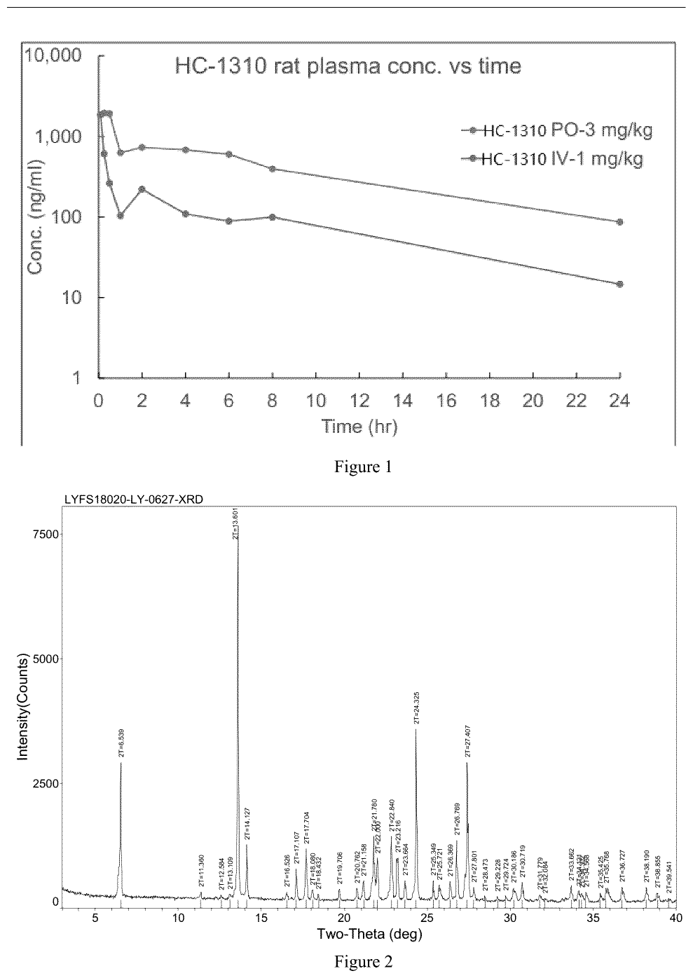

### DETAILED DESCRIPTION OF THE INVENTION

Various embodiments of this patent document provide crystalline forms of a quinoline compound, dosage forms the quinoline compound, methods of preparation thereof, and method of treating diseases with the dosage form.

Although the following contents may refer to or exemplify a specific embodiment of a dosage form, they are not limited to the specified ranges of the dosage form. In view of practicality and economy considerations, a person skilled in the art can make various modifications to, e.g., the amount of active ingredient and the dosage regimen of the dosage form for treating diseases or disorders.

Unless otherwise defined, all technical and scientific terms used herein have the same meaning as commonly understood by a person skilled in the field of the present invention. In case of conflict, the definitions provided in the application prevail.

The term “a”, “an” or “the” as used herein means “one or more” or “at least one”. That is, reference to any element or composition of the present invention by “a”, “an” or “the” does not exclude the possibility of the presence of a plurality of the elements and compositions. The term “extended release” or “ER” as used herein refers to extended release of an active pharmaceutical ingredient over an extended period of time, which is longer than about 2 hours, preferably longer than about 4 hours, more preferably longer than about 8 hours, more preferably longer than about 12 hours, more preferably longer than about 16 hours, or up to longer than about 24 hours.

The term “immediate release” or “IR” as used herein refers to release of more than or equal to about 80% of an active pharmaceutical ingredient in less than or equal to about 1 hour. Typically, more than or equal to about 85% or more than or equal to about 90% of an active pharmaceutical ingredient in an immediate release dosage form is released in less than or equal to about 1 hour, wherein the dissolution is determined using a USP type 1 dissolution system (Basket Apparatus) at 100 rpm and a temperature of 37±0.5° C. in a dissolution medium of 900 ml phosphate buffer solution at a pH of 6.8.

The term “pharmaceutically acceptable salts” means salts of compounds disclosed herein which are pharmaceutically acceptable, as defined above, and which possess the desired pharmacological activity. Non-limiting examples of such salts include base addition salts which may be formed when acidic protons present are capable of reacting with inorganic or organic bases. Non-limiting examples of acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “subject” refers to a mammal, and can be an animal or a human.

The term “treating” or “treatment” of any disease or condition refers, in some embodiments, to ameliorating the disease or disorder (i.e., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

An aspect of the patent document provides an extended release dosage form, which comprises:
an active ingredient comprising a therapeutically effective amount of a compound of Formula I, a solvate thereof, a prodrug thereof, or a pharmaceutically acceptable salt thereof, and
an extended release excipient,
wherein the active ingredient and the extended release excipient are in a ratio ranging from about 1:50 to about 1:5 by weight,
wherein the extended release excipient and its amount are selected that the dosage form provides a first Cₘₐₓ ranging from about 60% to about 85% of a second first Cₘₐₓ from an immediate release dosage form,
wherein the immediate release dosage form contains the same amount of the active ingredient as in the extended release dosage form,
wherein the compound of Formula I is represented as:

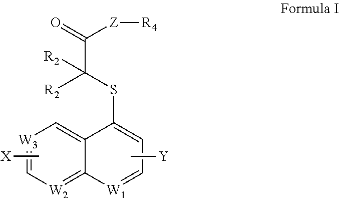

wherein:
Z is O, S or —NH—;
W₁ is N or CR^{a}; W₂ is N or CR^{b}; W₃ is N or CR^{c};
R^{a}, R^{b}, R^{c}, R₂, and R₃ are each independently selected from the group consisting of hydrogen, halogen, cyano, nitro, alkyl, alkenyl, alkynyl, cycloalkyl, heterocyclyl, aryl, heteroaryl, —OR^{d}, —S(O)ₘR^{d}, —C(O)R^{d}, C(O)OR^{d}, —C(O)NR^{e}R^{f}, —NR^{e}R^{f} and NR^{e}C(O)R^{f}, wherein said alkyl, alkenyl, alkynyl, cycloalkyl, heterocyclyl, aryl or heteroaryl are each independently and optionally further substituted with one or more of substituents selected from the group consisting of halogen, cyano, nitro, oxo, alkyl, haloalkyl, hydroxyalkyl, alkenyl, alkynyl, cycloalkyl, heterocyclyl, aryl, heteroaryl, —OR^{d}, —S(O)ₘR^{d}, —C(O)R^{d}, C(O)OR^{d}, —C(O)NR^{e}R^{f}, —NR^{e}R^{f} and NR^{e}C(O)R^{f};
R^{d} is selected from the group consisting of hydrogen, halogen, alkyl, cycloalkyl, heterocyclyl, aryl and heteroaryl, wherein said alkyl, cycloalkyl, heterocyclyl, aryl or heteroaryl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, carboxylic ester group, —C(O)NR^{e}R^{f}, —NR^{e}R^{f} and NR^{e}C(O)R^{f};
R^{e} and R^{f} are each independently selected from the group consisting of hydrogen, alkyl, cycloalkyl, heterocyclyl, aryl and heteroaryl, wherein said alkyl, cycloalkyl, heterocyclyl, aryl and heteroaryl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, and carboxylic ester group; and m is 0, 1, or 2;
X and Y are each independently selected from the group consisting of hydrogen, halogen, cyano, nitro, alkyl, cycloalkyl, haloalkyl and hydroxyalkyl;
provided that when Z is O or S, R₄ is selected from the group consisting of hydrogen, C₁-C₆ alkyl, and cycloalkyl, wherein said alkyl and cycloalkyl are each independently and optionally further substituted with one or more of substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, carboxylic ester group, —C(O)NR^{e}R^{f}, —NR^{e}R^{f} and NR^{e}C(O)R^{f}; and
provided that when Z is —NH—, R₄ is selected from the group consisting of hydrogen, aryl and heteroaryl, wherein R₄ is preferably pyridinyl.

The amount of the active ingredient in the dosage form, in some embodiments, ranges from about 1 to about 100, from about 1 to about 50, from about 1 to about 40, from about 1 to about 25, from about 1 to about 20, or from about 1 to about 10 mg. Nonlimiting examples of the amount of the active ingredient include about 1, about 2, about 5, about 8, about 10, about 15, about 20, about 25, about 30, about 40, about 50 mg, and any range between any two of the aforementioned values.

The dosage form may be in any suitable shape or configuration. Nonlimiting examples include pellets, balls, granules, globules, and tablets.

To achieve a desirable extended release, the active ingredient may be mixed with the extended release excipient, coated by the extended release excipient, or both. In some embodiments, the dosage form is a tablet with an extended release matrix, which is optionally coated with additional extended release excipient. In some embodiments, the dosage form is a tablet with the active ingredient being coasted with a layer of extended release excipient.

In some embodiments, the compound is

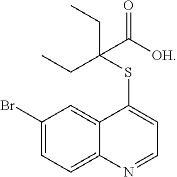 [SMILES: CCC(CC)(Sc1ccnc2ccc(Br)cc12)C(=O)O]

In some embodiments, the extended release excipient and its amount are selected that a single dose (administered once a day) of the dosage form at steady state provides a first Cₘₐₓ ranging from about 50% to about 95%, from about 60% to about 85%, from about 70% to about 80% or from about 60% to about 80% of a second first Cₘₐₓ from a single dose (administered once a day) of an immediate release dosage form at steady state. Nonlimiting examples of the first Cₘₐₓ relative to the second Cₘₐₓ include about 50%, about 60%, about 70%, about 75%, about 80%, about 85%, about 90%, and any range between any two of the aforementioned values.

In some embodiments, the extended release excipient and its amount are selected so that a single dose of the dosage form at steady state provides a first AUC ranging from about 70% to about 130%, from about 80% to about 120%, from about 90% to about 110% of a second first AUC from a single dose of the immediate release dosage form at steady state. Nonlimiting examples of the first AUC relative to the second AUC include about 75%, about 80%, about 85%, about 90%, about 95%, about 100%, about 110%, about 120%, and any range between any two of the aforementioned values.

In some embodiments, the active ingredient and the extended release excipient are in a ratio ranging from about 1:60 to about 1:5, from about 1:50 to about 1:10, from about 1:40 to about 1:20, or from about 1:40 to about 1:15 by weight. Nonlimiting examples of the ratio between the active ingredient and the extended release excipient include about 1:60, about 1:50, about 1:40, about 1:30, about 1:20, about 1:10, and any range between any two of the aforementioned values.

In some embodiments, the extended release excipient comprises HPMC. In some embodiments, the HPMC has a viscosity ranging from about 400 to about 1500, from about 400 to about 1200, from about 500 to about 1200, from about 500 to about 1100, or from about 600 to about 1000 millipascal seconds (mPa's) at room temperature when tested at a 2% concentration in a water solution. Unless otherwise specified, the viscosity of HPMC is tested at 2% concentration in a water solution.

In some embodiments, the extended release excipient comprises a high viscosity HPMC and a low viscosity HPMC. In some embodiments, the high viscosity HPMC has a viscosity ranging from about 2200 to about 6000, from about 2400 to about 5500, from about 2500 to about 5200, from about 2700 to about 5000, or from about 3000 to about 5000 mPa·s. In some embodiments, the low viscosity HPMC has a viscosity ranging from about 400 to about 1500, from about 400 to about 1300, from about 500 to about 1200, from about 500 to about 1000 mPa·s. In some embodiments, the ratio between the high viscosity HPMC and the low viscosity HPMC ranges from about 40:60 to about 60:40, from about 45:55 to about 65:35, from about 45:55 to about 55:45 by weight.

In some embodiments, the dosage form further comprises a filler comprising lactose monohydrate and microcrystalline cellulose. In some embodiments, the ratio between lactose monohydrate and microcrystalline cellulose ranges from about 10:1 to about 1:5, from about 10:1 to about 1:2, from about 10:1 to about 1:1, from about 10:1 to about 2:1, from about 10:1 to about 4:1, from about 10:1 to about 5:1, from about 10:1 to about 6:1, from about 10:1 to about 7:1, or from about 9:1 to about 6:1. In some embodiments, microcrystalline cellulose may be in various size ranging from about 30 μm to about 80 μm, from about 40 μm to about 80 μm, from about 40 μm to about 70 μm, from about 40 μm to about 60 μm, from about 45 μm to about 5 μm. Microcrystalline cellulose such as Avicel PH101 can be obtained from commercial sources.

In some embodiments, the dosage form is configured and the extended release excipient and its amount are selected so that when the dissolution profile is determined using a USP type 1 dissolution system (Basket Apparatus) at 100 rpm and a temperature of 37±0.5° C. in a dissolution medium of 900 ml phosphate buffer solution at a pH of 6.8, the active ingredient in the extended release dosage form has one or more or all of the following in vitro dissolution characteristics:
(a) in 1 hour, the released active ingredient accounts for about 1% to about 25%, about 1% to about 15%, about 1% to about 12%, about 1% to about 10%, about 1% to about 8%, about 1% to about 6%, about 1% to about 5%, about 1% to about 4%, about 1% to about 3%, about 1% to about 2%, about 5% to about 20%, about 5% to about 15%, about 5% to about 12%, about 5% to about 10%, about 5% to about 8%, about 5% to about 6%, about 5% to about 5%, about 5% to about 4%, about 5% to about 3%, about 1% to about 20%, about 1% to about 15%, about 1% to about 12%, about 1% to about 10%, about 1% to about 8%, about 1% to about 6%, about 1% to about 5%, about 1% to about 4%, or about 1% to about 3% of the total amount (w/w); Nonlimiting examples of the amount of active ingredient released within 1 hour include about 1%, about 2%, about 3%, about 4%, about 5%, about 6%, about 7%, about 8%, about 10%, about 12%, about 15%, about 18%, about 20%, about 23%, about 25%, and any range between any two of the aforementioned values.
(b) in 2 hours, the released active ingredient accounts for about 10% to about 40%, about 1% to about 30%, about 10% to about 25%, about 10% to about 20%, about 10% to about 15%, about 15% to about 30%, about 15% to about 25%, about 15% to about 20%, about 20% to about 30%, about 25% to about 30%, or about 20% to about 25%, of the total amount (w/w); Nonlimiting examples of the amount of active ingredient released within 2 hours include about 11%, about 12%, about 15%, about 18%, about 20%, about 22%, about 25%, about 30%, and any range between any two of the aforementioned values.
(c) in 4 hours, the released active ingredient accounts for about 30% to about 50%, about 35% to about 50%, about 35% to about 40%, or about 30% to about 35% of the total amount (w/w); Nonlimiting examples of the amount of active ingredient released within 4 hours include about 30%, about 35%, about 38%, about 40%, about 45%, about 50%, and any range between any two of the aforementioned values;
(d) in 6 hours, the released active ingredient accounts for about 30% to about 60%, about 40% to about 60%, about 45% to about 60%, or about 50% to about 60% of the total amount (w/w); Nonlimiting examples of the amount of active ingredient released within 6 hours include about 30%, about 35%, about 40%, about 45%, about 50%, about 55%, about 60%, and any range between any two of the aforementioned values;
(e) in 8 hours, the released active ingredient accounts for about 50% to about 75%, about 50% to about 70%, about 60% to about 70%, or about 65% to about 70% of the total amount (w/w); Nonlimiting examples of the amount of active ingredient released within 8 hours include about 50%, about 55%, about 60%, about 65%, about 70%, about 75%, and any range between any two of the aforementioned values;
(f) in 12 hours, the released active ingredient accounts for about 70% to about 95%, about 70% to about 90%, about 70% to about 85%, or about 70% to about 80% of the total amount (w/w); Nonlimiting examples of the amount of active ingredient released within 12 hours include about 70%, about 75%, about 80%, about 85%, about 90%, about 95%, and any range between any two of the aforementioned values;
(g) in 16 hours, the released active ingredient accounts for about 80% to about 100%, about 80% to about 100%, about 85% to about 100%, or about 85% to about 90% of the total amount (w/w); Nonlimiting examples of the amount of active ingredient released within 16 hours include about 80%, about 85%, about 90%, about 95%, about 100%, and any range between any two of the aforementioned values;
(h) in 20 hours, the released active ingredient accounts for about 85% to about 100%, about 90% to about 100%, about 95% to about 100%, or about 85% to about 95% of the total amount (w/w); Nonlimiting examples of the amount of active ingredient released within 20 hours include about 85%, about 90%, about 95%, about 100%, and any range between any two of the aforementioned values;
and/or
(i) in 24 hours, the released active ingredient accounts for about 90% to about 100%, about 95% to about 100%, or about 98% to about 100% of the total amount (w/w); Nonlimiting examples of the amount of active ingredient released within 24 hours include about 90%, about about 95%, about 100%, and any range between any two of the aforementioned values;

In some embodiments, the dosage form has any one, two, three, four, five, six, seven or eight of the above dissolution characteristics. In some embodiments, the pharmaceutical composition or dosage form provides the dissolution of the active ingredient having the above (a). In some embodiments, the pharmaceutical composition or dosage form provides the dissolution of the active ingredient having the above (a) and (b). In some embodiments, the pharmaceutical composition or dosage form provides the dissolution of the active ingredient having the above (a), (b) and (e). In some embodiments, the pharmaceutical composition or dosage form provides the dissolution of the active ingredient having the above (a), (b), (f) and (i). In some embodiments, the pharmaceutical composition or dosage form provides the dissolution of the active ingredient having the above (a), (b), (f), (g) and (h). In some embodiments, the pharmaceutical composition or dosage form provides the dissolution of the active ingredient having the above (a), (b), (f), (g) and (i). In some embodiments, the pharmaceutical composition or dosage form provides the dissolution of the active ingredient having the above (a), (b), (f), (g), (h), and (i).

Another aspect provides a method of treating a disease in subject, comprising administering to the subject a dosage form disclosed herein. In some embodiments, the disease is selected from gout, gout attack, gouty arthritis, hyperuricemia, hypertension, cardiovascular diseases, coronary artery disease, Lesch-Nyhan syndrome, Kearns-Sayre Syndrome, nephropathy, kidney stone, renal failure, joint inflammation, arthritis, urolithiasis, lead poisoning, hyperparathyroidism, psoriasis, and sarcoidosis or hypoxanthine-guanine phosphoribosyl transferase deficiency disease. In some embodiments, the dosage form is administer prophylactically.

### EXAMPLES

### Example 1. Preparation of Crystalline Form I of Quinoline Compounds

59.2 kg of anhydrous methanol was added to a tank reactor, and then stirred. The reaction system was heated to reflux, to which was added 1135 g of crude 2-[4-(6-bromoquinolyl)thio]-2-ethylbutyric acid, and then heated to 65-70° C. to dissolve; the resultant solution was filtered to remove insoluble substances, and then the filtrate was transferred to the tank reactor, followed by heating the reaction system to 65-70° C. to dissolve and allowing the solution to become clear. The heating was turned off, and the solution was naturally cooled for crystallization for 20-24 h. The temperature of the reaction system was controlled to be 15-20° C., and the system was stirred for crystallizing for 2 h. The system was filtered and dried to obtain the product crystalline form I of compound 2-[4-(6-bromoquinolyl)-thio]-2-ethylbutyric acid as off-white to white crystalline granules;

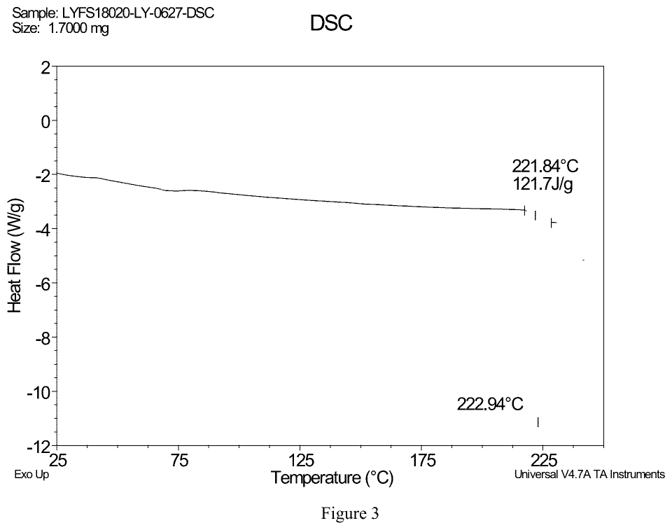

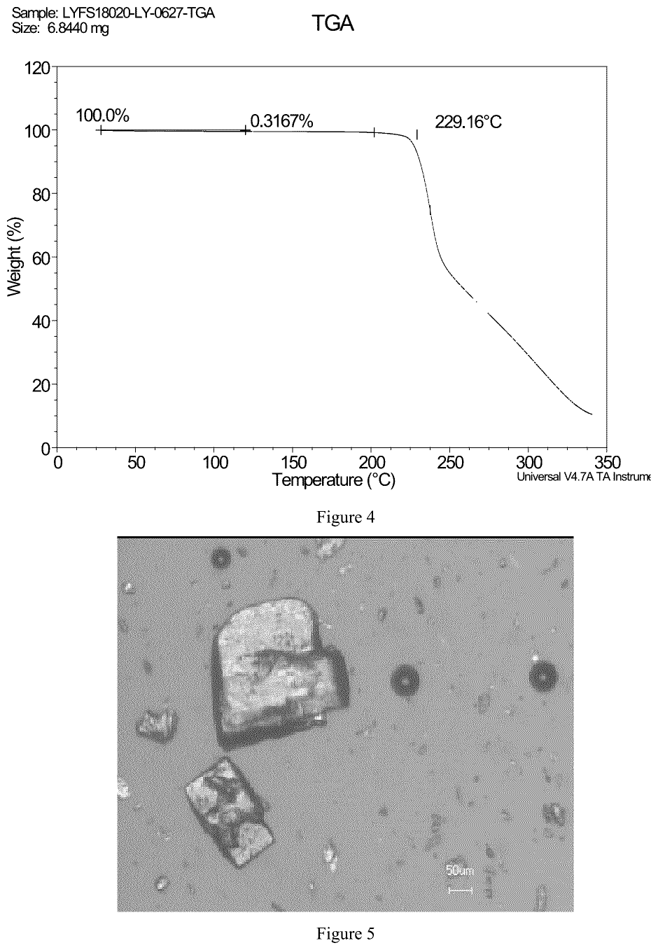

XRD, DSC, TGA, and PLM spectra of crystalline form I are shown in FIGS. 2 to 5.

### Example 2. Preparation of Crystalline Form II of Quinoline Compounds

About 10 mg of crude 2-[4-(6-bromoquinolyl)-thio]-2-ethylbutyric acid was added to 0.7 mL of tetrahydrofuran and 0.4 mL of dichloromethane, and then the solution was allowed to dissolve and become clear, followed by filtering. The filtrate was placed in a fume hood, and evaporated in an open state at room temperature to obtain the product of crystalline form II of 2-[4-(6-bromoquinolyl)-thio]-2-ethylbutyric acid;

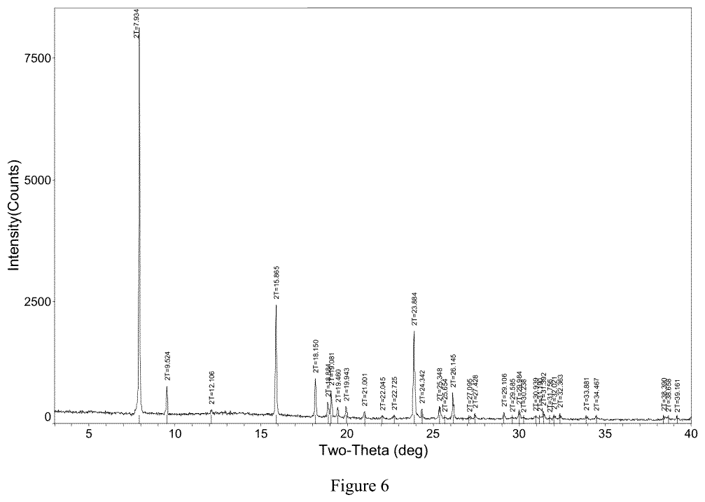

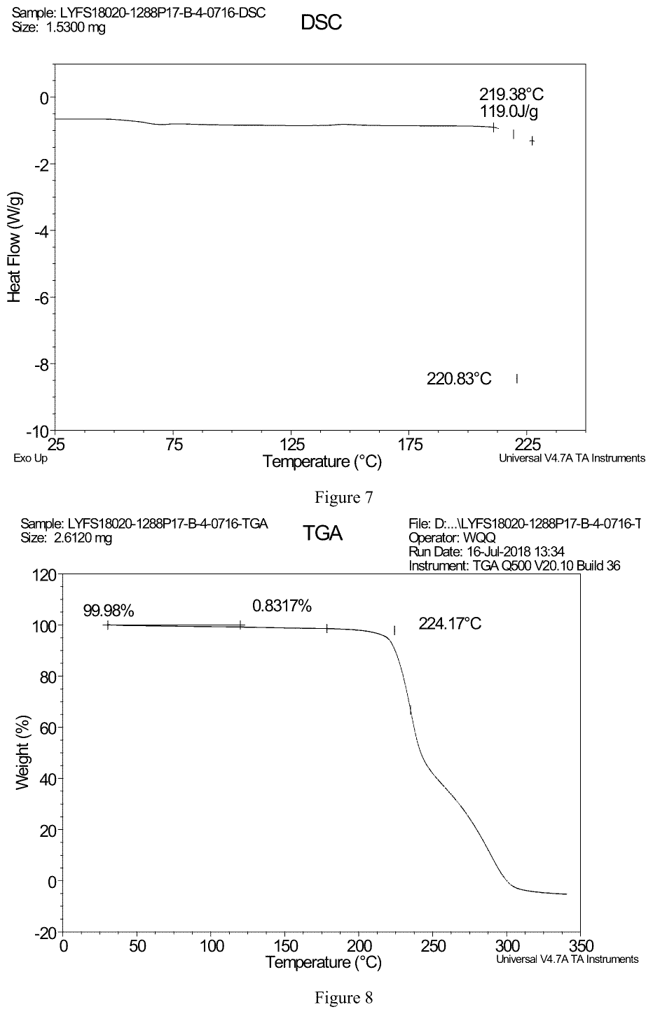

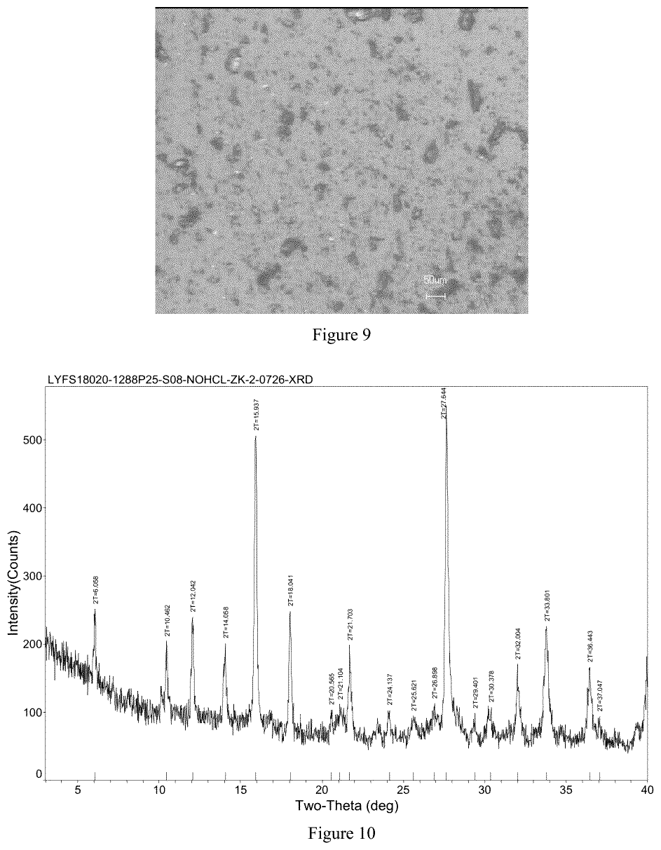

XRD, DSC, TGA, and PLM spectra of crystalline form II are shown in FIGS. 6 to 9.

### Example 3. Preparation of Sodium Salt Crystalline Form of Quinoline Compounds

About 300 mg of crude 2-[4-(6-bromoquinolyl)-thio]-2-ethylbutyric acid was added into 12 mL of water, and then stirred, to which was added 1 mol/L of NaOH aqueous solution dropwise to pH 12. The sample was allowed to precipitate under stirring. After stirring for additional 3 min, the solution was filtered and dried overnight in vacuum, to obtain the sodium salt crystalline form of compound 2-[4-(6-bromoquinolyl)-thio]-2-ethylbutyric acid;

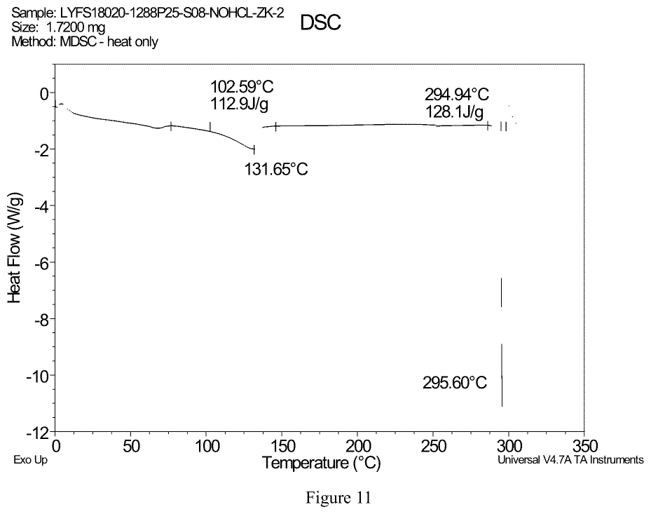

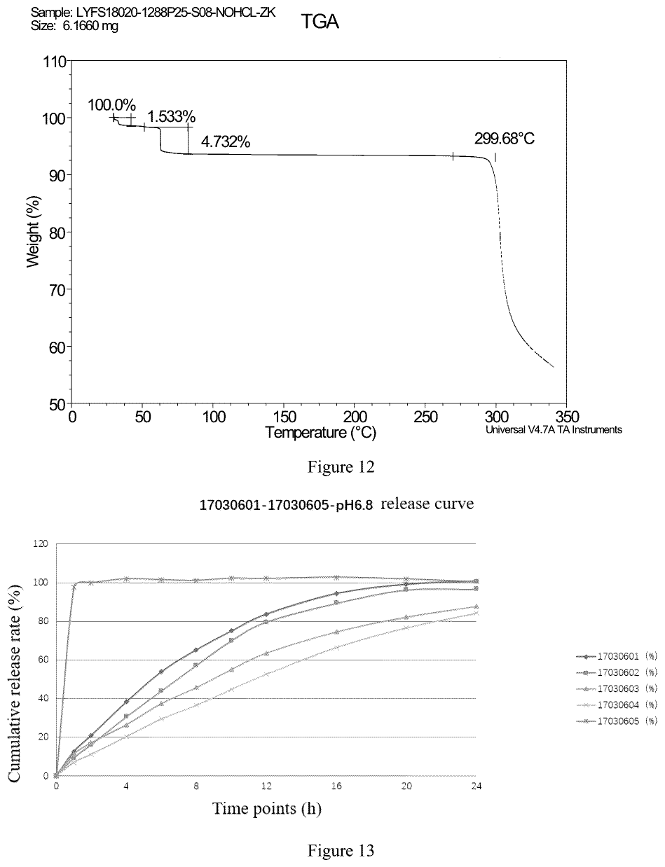

XRD, DSC, and TGA spectra of sodium salt crystalline form are shown in FIGS. 10 to 12.

### Example 4. Preparation of Quinoline Compound Sustained-Release Tablets

Formula: 5 g of crystalline form I of compound 2-[4-(6-bromoquinolyl)-thio]-2-ethylbutyric acid prepared in Example 1, 226.1 g of lactose monohydrate, 30 g of microcrystalline cellulose PH101, 57 g of hydroxypropyl methylcellulose K4M (HPMC K4M), 60 g of hydroxypropyl methylcellulose K100LV (HPMC K100LV), and 1.9 g of magnesium stearate.

Preparation method:
a. The raw and adjuvant materials were weighed according to the ratio, and lactose was passed through a 60-mesh sieve;
b. ⅓ of the pre-determined amount of lactose in the formula was placed in a wet granulator, stirred for 5 min, and then ⅓ of the pre-determined amount of lactose and ½ of the pre-determined amount of quinoline compound were added and stirred for 5 min. Finally, the remaining amounts of lactose and quinoline compounds were added, and stirred for 5 min. Then, microcrystalline cellulose and hydroxypropyl methylcellulose were added and stirred for 10 min;
c. Water was added to make soft materials, the granulation process was carried out by passing through a 20-mesh sieve, the materials were dried at 60° C. to 3% moisture content, and then the sorting was performed with a 20-mesh sieve;
d. Magnesium stearate was mixed with the granules obtained in step c, and then pressed into tablets. The tablets were coated at a weight gain of 3%, to obtain the sustained-release tablets.

The beneficial effects of the present invention were further demonstrated by reference to the following Experimental examples:

### Experiment Example 1: Comparison of Different Ratios and Types of Sustained-Release Materials Used for Quinoline Compound Sustained-Release Tablets

### 1. Formula

TABLE 1
Release comparison of different ratios and types of sustained-release materials
used in quinoline compound sustained-release tablets (specification: 5 mg).

| Components | 17030601 | 17030602 | 17030603 | 17030604 | 17030605 |
|---|---|---|---|---|---|
| (100 tablets) | (mg/tablet) | (mg/tablet) | (mg/tablet) | (mg/tablet) | (mg/tablet) |
| API | 5 | 5 | 5 | 5 | 5 |
| HPMC K750 | 120 | 160 | — | — | — |
| HPMC K4M | — | — | 120 | 160 | — |
| HPC HXF | — | — | — | — | 120 |
| (Hydroxypropyl | | | | | |
| cellulose HXF) | | | | | |
| Lactose monohydrate | 255 | 215 | 255 | 215 | 255 |
| 10% PVPK30 | 141.6 | 139.8 | 142.8 | 140.3 | 49.6 |
| aqueous solution | | | | | |
| Magnesium stearate | 4 | 4 | 4 | 4 | 4 |

### 2. Preparation Steps:

1) 10.00 g of PVP K30 was weighed and added into 90.00 g of purified water, and then stirred to dissolve. Thus, 10% of PVPK30 aqueous solution was prepared for later use.
2) Based on the preparation of 100 tablets, the prescribed amounts of API and lactose monohydrate were weighed, and well mixed through an 80 mesh sieve using an incremental analysis.
3) The prescribed amount of sustained-release materials was weighed and mixed with the aforementioned powder through sieving (60-mesh).
4) A suitable amount of adhesives was added to make soft materials, and 20-mesh sieve was used for granulation; the granules were dried at 60° C. for 1 h, and then sorted through a 20-mesh sieve.
5) Magnesium stearate was added in a ratio of 1% by weight of the granules and mixed well.
6) Using a rotary tablet machine, 10 mm of shallow arc circular punches were used for pressing tablets.

### 3. Release Detection:

Medium: pH 6.8 phosphate buffer Rolling basket method: 100 RPM

The release rates of 3 tablet samples for each formula were tested, and then the average value was calculated. Samples were taken at 1 h, 2 h, 4 h, 6 h, 8 h, 10 h, 12 h, 16 h, 20 h, and 24 h, and subjected to HPLC analysis to obtain the release curve.

### 4. Results

### See Table 2 and FIG. 13.

TABLE 2
The release curves of the samples of quinoline compound sustained-release tablets
(specification: 5 mg) for screening of sustained-release materials types and different ratios.

| Formula | | 1 h | 2 h | 4 h | 6 h | 8 h | 10 h | 12 h | 16 h | 20 h | 24 h |
|---|---|---|---|---|---|---|---|---|---|---|---|
| 17030601 | The cumulative release | 12.5 | 20.9 | 38.2 | 53.8 | 65.2 | 74.9 | 83.6 | 94.3 | 99.0 | 100.8 |
| | rate (%) | | | | | | | | | | |
| | RSD (%) | 16.92 | 18.25 | 18.78 | 19.06 | 15.88 | 11.36 | 8.69 | 5.29 | 1.49 | 0.52 |
| 17030602 | The cumulative release | 9.1 | 16.1 | 30.5 | 43.9 | 57.2 | 69.9 | 79.6 | 89.3 | 96.2 | 96.6 |
| | rate (%) | | | | | | | | | | |
| | RSD (%) | 10.82 | 4.14 | 1.92 | 3.09 | 4.55 | 6.11 | 6.71 | 3.30 | 1.84 | 0.20 |
| 17030603 | The cumulative release | 11.1 | 17.0 | 26.4 | 37.4 | 45.7 | 55.1 | 63.6 | 74.6 | 82.2 | 87.8 |
| | rate (%) | | | | | | | | | | |
| | RSD (%) | 14.56 | 6.67 | 1.62 | 0.05 | 2.63 | 2.09 | 1.31 | 0.14 | 0.52 | 0.31 |
| 17030604 | The cumulative release | 6.8 | 11.1 | 20.2 | 29.4 | 36.5 | 44.8 | 52.6 | 66.4 | 76.6 | 84.1 |
| | rate (%) | | | | | | | | | | |
| | RSD (%) | 6.30 | 3.11 | 3.51 | 3.34 | 3.16 | 3.46 | 2.74 | 3.91 | 5.26 | 4.58 |
| 17030605 | The cumulative release | 97.5 | 99.9 | 102.0 | 101.5 | 101.2 | 102.3 | 102.2 | 102.8 | 101.9 | 100.5 |
| | rate (%) | | | | | | | | | | |
| | RSD (%) | 0.16 | 0.48 | 0.22 | 0.22 | 0.49 | 0.30 | 0.08 | 0.39 | 0.15 | 0.51 |

The results indicated that the tablets prepared with sustained-release material HPC HXF had no delayed-release effect in a medium at pH 6.8, with a release rate of over 90% after 1 h. The tablets comprising slow-release materials HPMC K4M at a ratio of 30% and 40% released slowly in the medium at pH 6.8, with a release rate of less than 90% after 24 h. The release rate of the tablets comprising 30% and 40% of sustained-release materials HPMC K750 was moderate, with a release rate of over 95% after 16-20 hours.

### Experimental Example 2: The Amounts of Sustained-Release Materials in Quinoline Compound Sustained-Release Tablets

### 1. Formula

TABLE 3
Comparison of different amounts of sustained-release
materials used in quinoline compound sustained-
release tablets (specification: 5 mg).

| | | | 17050401 | 17042501 |
|---|---|---|---|---|
| Components | | | (mg/tablet) | (mg/tablet) |
| API | | | 5 | 5 |
| HPMC K750 | | | 115 | 160 |
| Lactose monohydrate | | | 255 | 215 |
| 75% ethanol solution | | | 216.9 | 253.4 |
| Magnesium stearate | | | 3.8 | 3.8 |
| Coating solution | Opadry | 5% | Weight gain | Weight gain |
| | (21K58794) | | 3.3% | 3.3% |

### 2. Preparation Steps:

1) The prescribed amounts of API and lactose were weighed, and well mixed through a 60-mesh sieve using an incremental analysis.
2) The prescribed amount of sustained-release materials was weighed and mixed with the aforementioned powder through sieving for three times.
3) 75% ethanol solution was added to make soft materials, and 20-mesh sieve was used for granulation. The granules were dried at 50° C. for 1 h, and then sorted through a 20-mesh sieve.
4) Magnesium stearate was added in a ratio of 1% by weight of the granules and mixed well.
5) Using a rotary tablet machine, 10 mm of shallow arc circular punches were used for pressing tablets.
6) The coating solution was prepared according to the ratio in the formula, and thus a coating solution at a content of 5% was obtained.
7) A BY300A small coating machine was used for coating, and a certain amount of plain tablets were weighed and placed in a coating pot. The coating was carried out at a weight increase of 3% to prepare the tablets.

### 3. Detection of Release Curve:

Medium: pH 6.8 phosphate buffer Rolling basket method: 100 RPM

For each plain or coated tablet, the release rates of 3 tablet samples were tested, and then the average value was calculated. Samples were taken at 1 h, 2 h, 4 h, 16 h, 20 h, and 24 h, and subjected to HPLC analysis to obtain the release curve.

### 4. Results

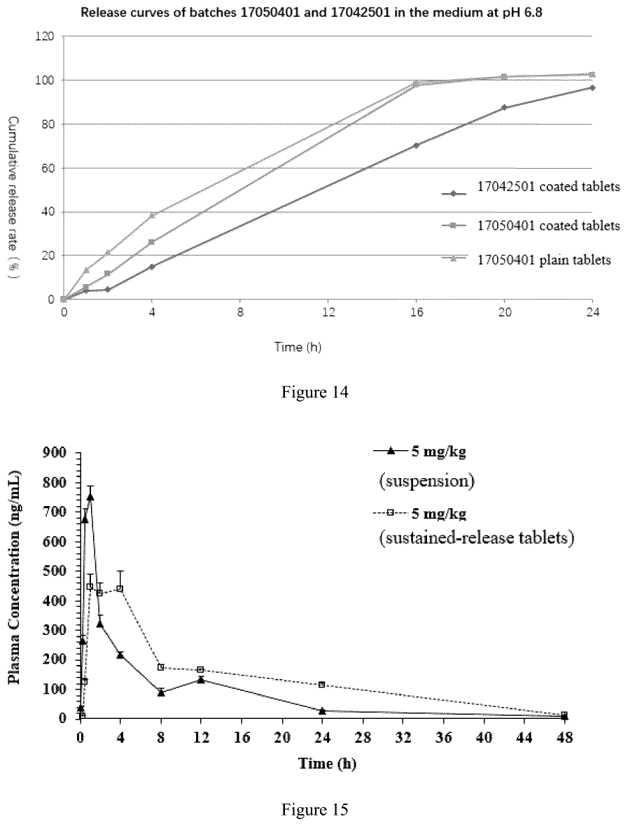

See Table 4 and FIG. 14.

TABLE 4
Release data of coated and plain tablets for optimizing the amounts of sustained-
release materials in quinoline compound sustained-release tablets (specification: 5 mg).

| Formula | | 1 h | 2 h | 4 h | 16 h | 20 h | 24 h |
|---|---|---|---|---|---|---|---|
| 17042501 | The cumulative release rate (%) | 4.0 | 4.4 | 15.0 | 70.3 | 87.4 | 96.5 |
| coated tablets | RSD (%) | 13.17 | 14.35 | 3.96 | 3.92 | 3.35 | 3.02 |
| 17050401 | The cumulative release rate (%) | 5.5 | 11.5 | 26.1 | 97.7 | 101.4 | 102.4 |
| coated tablets | RSD (%) | 15.39 | 5.96 | 3.27 | 2.56 | 1.11 | 0.78 |
| 17050401 | The cumulative release rate (%) | 13.4 | 21.4 | 38.4 | 98.9 | 101.7 | 102.9 |
| plain tablets | RSD (%) | 20.52 | 19.08 | 12.78 | 0.34 | 1.02 | 0.85 |

According to the data in Table 4 and FIG. 14, after reducing the amounts of the sustained-release materials, the release of coated tablets and plain tablets both reached over 95% within 16 hours, approaching complete release. The release of coated tablets was about 10% lower than that of plain tablets in the early stage, but there was almost no difference in release after 16 hours.

### Experimental Example 3: Animal Experiment

According to the formula of batch 17050401, the amounts were increased to 1000 tablets, and then 18071003 batch samples were produced and subjected to PK testing in beagle dogs. The experimental design was as follows:

The experiment was divided into two stages, and 6 beagles were included, with half male and half female. In the first stage, the experimental animals were orally administered the suspension of active ingredients at a single dose of 5 mg/kg, in which the vehicle was 0.5% sodium carboxymethyl cellulose (0.5% CMC-Na) aqueous solution. After a 7-day washout period, in the second stage, the same experimental animals were orally administered 10 quinoline compound sustained-release tablets (specification 5 mg) once. All animals were fasted overnight before administration and resumed feeding approximately 4 h after administration. 75 mL of KCl-HCl buffer was administered via a gastric tube 15 min before administration. Plasma samples of animals were collected prior to administration as well as at 0.0833 h, 0.25 h, 0.5 h, 1 h, 2 h, 4 h, 8 h, 12 h, 24 h, and 48 h after administration. The concentration of quinoline compounds in plasma samples was determined by the validated LC-MS/MS method. The in vivo experimental results in beagle dogs administered with 5 mg/kg of suspension and 5 mg of tablets are shown in Table 5 and FIG. 15.

TABLE 5
The average pharmacokinetic parameters of quinoline compound
suspension or sustained-release tablets in male and
female beagle dogs after administration (n = 6).

| | Form of preparation | | | |
|---|---|---|---|---|
| | Suspension | | Sustained-release tablets | |
| | Route of administration | | | |
| | PO | | PO | |
| | Dosage (mg/kg) | | | |
| Pharmacokinetic | 5.0 | | 5.0 | |
| parameters | Mean | SD | Mean | SD |
| Cₘₐₓ (ng/mL) | 827 | 420 | 620 | 140 |
| Tₘₐₓ (h) | 1.08 | 0.492 | 2.17 | 1.47 |
| T_{1/2} (h) | 8.44 | 3.59 | 8.99 | 2.42 |
| AUC₀₋ₗₐₛₜ (h · ng/mL) | 3520 | 802 | 5842 | 1042 |
| AUC_{0-inf} (h · ng/mL) | 3655 | 884 | 5968 | 1304 |

The experimental results showed that under the conditions of ensuring similar AUC, the Cₘₐₓ of sustained-release tablets was significantly reduced by nearly 25%, that could effectively prevent the incidence of adverse reactions caused by excessive plasma concentrations.

Since HPMC K750, used as a sustained-release material in the tablet formulation, was found to account for a large proportion in the scale-up production, the wear resistance of the tablets during coating was insufficient, the yield was low, and the process compliance decreased. Thus, HPMC K750 was substituted with a mixture of HPMC K4M and K100LV, that not only overcame the problems in the preparation process of tablets, but also obtained the same extended-release effect and pharmacokinetic parameters as those of slow-release tablets in which HPMC K750 was used as sustained-release materials. Therefore, stability studies were carried out on tablet formulations using HPMC K4M and K100LV as sustained-release materials in the subsequent experiment.

### Experimental Example 4. Stability Study of Quinoline Compound Sustained-Release Tablets of the Present Invention

### 1. Formula

See Table 6.

TABLE 6
Formulation of quinoline compound sustained-
release tablets (specification: 5 mg).

| | | | 19082901 |
|---|---|---|---|
| Components | | | (mg/tablet) |
| API | | | 5 |
| HPMC K4M | | | 57 |
| HPMC K100LV | | | 60 |
| Lactose monohydrate | | | 226.1 |
| Microcrystalline cellulose PH101 | | | 30 |
| Magnesium stearate | | | 1.9 |
| Coating solution | Opadry (21K58794) | 5% | Weight gain 3% |

### 2. Preparation Steps:

1) The prescribed amount of lactose was weighed and passed through a 60-mesh sieve.
2) ⅓ of the prescibed amount of lactose was placed in a wet granulator, stirred for 5 min, and then ⅓ of the pre-determined amount of lactose and ½ of the pre-determined amount of quinoline compound in the formula were added and stirred for 5 min. Finally, the remaining amounts of quinoline compounds and lactose were added, and stirred for additional 5 min. Then, the prescribed amounts of HMPC K4M, HPMCK100Lv, and microcrystalline cellulose PH 101 were addeded and stirred for 10 min.
3) Some water was added to make soft materials, the granulation process was performed by passing through a 20-mesh sieve. The materials were dried at 60° C. to the moisture content of <4%, and then the sorting was carried out with a 20-mesh sieve.
4) Magnesium stearate was added in a ratio of 0.5% by weight of the granules and mixed well.
5) Using a rotary tablet machine, 10 mm of shallow arc circular punches were used for pressing tablets.
6) The coating solution was prepared according to the ratio in the formula, and thus a coating solution at a content of 12% was obtained.
7) A BY300A small coating machine was used for coating, and a certain amount of plain tablets were weighed and placed in a coating pot. The coating was carried out at a weight increase of 3% to prepare the tablets.

### 3. The Stability Results are Shown in Table 7.

TABLE 7
Investigation on the stability of sample (19082901) obtained after optimizing
the formula of 5 mg preparation.
Accelerated conditions: 40° C. ± 2° C./75% RH ± 5% RH
Long-term conditions: 25° C. ± 2° C./60% RH ± 10% RH

| | | | | 1 month | 2 months | 3 months | 3 months |
|---|---|---|---|---|---|---|---|
| | | | | under | under | under | under |
| | | | Day | accelerated | accelerated | accelerated | long-term |
| Inspection | Time points (h) | | 0 | conditions | conditions | conditions | conditions |
| items | 0 | | 0 | 0 | 0 | 0 | 0 |
| Release rate | 1 | | 7.4 | 7.4 | 7.5 | 7.2 | 7.2 |
| (%) | 2 | | 15.2 | 14.8 | 15.0 | 14.8 | 14.8 |
| | 4 | | 31.7 | 30.1 | 30.0 | 30.5 | 30.8 |
| | 6 | | 48.2 | 45.8 | 45.0 | 45.8 | 46.1 |
| | 8 | | 63.8 | 59.8 | 59.8 | 59.9 | 60.0 |
| | 10 | | 76.0 | 71.4 | 70.8 | 71.3 | 71.3 |
| | 12 | | 85.9 | 81.2 | 81.0 | 81.8 | 80.7 |
| | 16 | | | 98.4 | 95.7 | 96.6 | 95.3 |
| | 20 | | 103.0 | 100.4 | 102.4 | 101.3 | 102.1 |
| | 24 | | 103.0 | 100.9 | 103.3 | 101.0 | 102.8 |
| Related | Single | RRT0.37 | ND | ND | ND | ND | ND |
| substances | foreign | API-ZA06 | 0.06 | 0.06 | 0.06 | 0.06 | 0.06 |
| (%) | material | (RRT0.94) | | | | | |
| | ≤0.5% | API-ZA05 | 0.03 | 0.02 | 0.04 | 0.04 | 0.03 |
| | | (RRT1.82) | | | | | |
| | | IM2 | 0.03 | 0.03 | 0.03 | 0.03 | 0.03 |
| | | (RRT2.02) | | | | | |

| | | | | | |
|---|---|---|---|---|---|
| All foreign materials | 0.12 | 0.11 | 0.13 | 0.13 | 0.11 |
| ≤2.0% | | | | | |

The results indicated that the extended-release effects of the tablets using HPMC K4M and K100LV as sustained-release materials are consistent with those using HPMC K750 as sustained-release materials. In both accelerated and long-term experiments, there was no growth trend in the substances related to the sustained-release tablets of the present invention, and the release curve remained stable and unchanged, demonstrating that the product was stable.

1. An extended release dosage form, comprising
an active ingredient comprising a therapeutically effective amount of a compound of Formula I, a solvate thereof, a prodrug thereof, or a pharmaceutically acceptable salt thereof, and
an extended release excipient,
wherein the active ingredient and the extended release excipient are in a ratio ranging from about 1:50 to about 1:5 by weight,
wherein the extended release excipient and its amount are selected so that the dosage form, when administered once a day, provides a first Cₘₐₓ ranging from about 60% to about 85% of a second first Cₘₐₓ from an immediate release dosage form administered once a day,
wherein the immediate release dosage form contains the same amount of the active ingredient as in the extended release dosage form,
wherein the compound of Formula I is represented as: 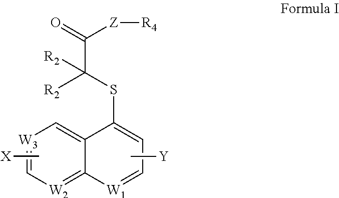
wherein:
Z is O, S or —NH—;
W₁ is N or CR^{a}; W₂ is N or CR^{b}; W₃ is N or CR^{c};
R^{a}, R^{b}, R^{c}, R₂, and R₃ are each independently selected from the group consisting of hydrogen, halogen, cyano, nitro, alkyl, alkenyl, alkynyl, cycloalkyl, heterocyclyl, aryl, heteroaryl, —OR^{d}, —S(O)ₘR^{d}, —C(O)R^{d}, C(O)OR^{d}, —C(O)NR^{e}R^{f}, —NR^{e}R^{f} and NR^{e}C(O)R^{f}, wherein said alkyl, alkenyl, alkynyl, cycloalkyl, heterocyclyl, aryl or heteroaryl are each independently and optionally further substituted with one or more of substituents selected from the group consisting of halogen, cyano, nitro, oxo, alkyl, haloalkyl, hydroxyalkyl, alkenyl, alkynyl, cycloalkyl, heterocyclyl, aryl, heteroaryl, —OR^{d}, —S(O)ₘR^{d}, —C(O)R^{d}, C(O)OR^{d}, —C(O)NR^{e}R^{f}, —NR^{e}R^{f} and NR^{e}C(O)R^{f};
R^{d} is selected from the group consisting of hydrogen, halogen, alkyl, cycloalkyl, heterocyclyl, aryl and heteroaryl, wherein said alkyl, cycloalkyl, heterocyclyl, aryl or heteroaryl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, carboxylic ester group, —C(O)NR^{e}R^{f}, —NR^{e}R^{f} and NR^{e}C(O)R^{f};
R^{e} and R^{f} are each independently selected from the group consisting of hydrogen, alkyl, cycloalkyl, heterocyclyl, aryl and heteroaryl, wherein said alkyl, cycloalkyl, heterocyclyl, aryl and heteroaryl are each independently and optionally further substituted with one or more of the substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, and carboxylic ester group; and m is 0, 1, or 2;
X and Y are each independently selected from the group consisting of hydrogen, halogen, cyano, nitro, alkyl, cycloalkyl, haloalkyl and hydroxyalkyl;
provided that when Z is O or S, R₄ is selected from the group consisting of hydrogen, C₁-C₆ alkyl, and cycloalkyl, wherein said alkyl and cycloalkyl are each independently and optionally further substituted with one or more of substituents selected from the group consisting of halogen, cyano, nitro, hydroxy, oxo, alkyl, haloalkyl, hydroxyalkyl, alkoxy, cycloalkyl, heterocyclyl, aryl, heteroaryl, carboxyl, carboxylic ester group, —C(O)NR^{e}R^{f}, —NR^{e}R^{f} and NR^{e}C(O)R^{f};
provided that when Z is —NH—, R₄ is selected from the group consisting of hydrogen, aryl and heteroaryl, and preferably pyridinyl.

2. The dosage form of claim 1, wherein the compound is

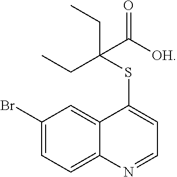 [SMILES: CCC(CC)(Sc1ccnc2ccc(Br)cc12)C(=O)O]

3. The dosage form of claim 1, wherein the extended release excipient and its amount are selected so that the dosage form provides a first AUC ranging from about 70% to about 130% of a second first AUC from the immediate release dosage form.

4. The dosage form of claim 1, wherein the extended release excipient and its amount are selected so that the dosage form has an in-vitro dissolution according to the following:
(a) from about 5% to about 20% in 1 hour,
(b) from about 10% to about 30% in 2 hours,
(c) from about 35% to about 40% in 4 hours,
(d) from about 60% to about 85% in 10 hours, and
(e) from about 90% to about 100% in 24 hours,
wherein the in-vitro dissolution is evaluated with USP type 1 dissolution system (Basket Apparatus) at 100 rpm and a temperature of 37±0.5° C. in a dissolution medium of 900 ml phosphate buffer solution at a pH of 6.8.

5. The dosage form of claim 1, wherein the active ingredient and the extended release excipient are in a ratio ranging from about 1:40 to about 1:15 by weight.

6. The dosage form of claim 1, wherein the extended release excipient comprises HPMC.

7. The dosage form of claim 6, wherein the HPMC has a viscosity ranging from about 400 to about 1200 millipascal seconds (mPa·s) at room temperature.

8. The dosage form of claim 6, wherein the extended release excipient comprises a high viscosity HPMC and a low viscosity HPMC, wherein the high viscosity HPMC has a viscosity ranging from about 2200 to about 5500 mPa's and the low viscosity HPMC has a viscosity ranging from about 400 to about 1500 mPa·s.

9. The dosage form of claim 8, wherein the high viscosity HPMC and the low viscosity HPMC are in a ratio ranging from about 40:60 to about 60:40 by weight.

10. The dosage form of claim 1, further comprising a filler comprising lactose monohydrate and microcrystalline cellulose in a ratio ranging from about 10:1 to about 1:5.

11. The dosage form of claim 1, further comprising a lubricant selected from the group consisting of magnesium stearate, sodium fumarate, glyceryl behenate, and stearic acid.

12. The dosage form of claim 1, wherein the compound is 2-[4-(6-bromoquinolyl)thio]-2-ethylbutyric acid which has an X-ray powder diffraction (XRPD) pattern comprising peaks at diffraction angle 2θ values comprising 6.5±0.2°, 13.6±0.2°, 14.1±0.2°, 17.7±0.2°, 21.8±0.2°, 22.0±0.2°, 22.8-0.2°, 23.2±0.2°, 24.3±0.2°, 26.8±0.2°, and 27.4±0.2° when irradiated with a Cu-Kα light source (Form I).

13. The dosage form of claim 12, wherein relative intensity of the characteristic peaks are as follows

| Diffraction angle 2θ | Relative intensity % |
|---|---|
| 6.5 ± 0.2 | 36.1 |
| 13.6 ± 0.2 | 100.0 |
| 14.1 ± 0.2 | 14.1 |
| 17.7 ± 0.2 | 13.6 |
| 21.8 ± 0.2 | 17.0 |
| 22.0 ± 0.2 | 11.1 |
| 22.8 ± 0.2 | 16.9 |
| 23.2 ± 0.2 | 11.1 |
| 24.3 ± 0.2 | 45.6 |
| 26.8 ± 0.2 | 15.7 |
| 27.4 ± 0.2 | 36.5. |

14. The dosage form of claim 1, wherein the compound is 2-[4-(6-bromoquinolyl)thio]-2-ethylbutyric acid which has an X-ray powder diffraction (XRPD) pattern comprising peaks at diffraction angle 2θ values comprising 7.9±0.2°, 9.5±0.2°, 15.9±0.2°, 18.2±0.2°, 19.1±0.2°, 23.9±0.2°, and 26.1±0.2° when irradiated with a Cu-Kα light source (Form II).

15. The dosage form of claim 1, wherein the compound is sodium salt of 2-[4-(6-bromoquinolyl)thio]-2-ethylbutyric acid which has an X-ray powder diffraction (XRPD) pattern comprising peaks at diffraction angle 2θ values comprising 6.1±0.2°, 10.5±0.2°, 12.0±0.2°, 14.1±0.2°, 15.9±0.2°, 18.0±0.2°, 21.7±0.2°, 27.6±0.2°, 32.0±0.2°, 33.8±0.2°, and 36.4±0.2° when irradiated with a Cu-Kα light source (sodium salt).

16. The dosage form of claim 15, wherein relative intensity of the characteristic peaks are as follows

| | |
|---|---|
| 27.6 ± 0.2 | 100.0 |
| 32.0 ± 0.2 | 22.0 |
| 33.8 ± 0.2 | 34.2 |
| 36.4 ± 0.2 | 22.4. |

17. A method of treating a disease in subject, comprising administering to the subject a dosage form of claim 1, wherein the disease is selected from the group consisting of gout, gout attack, gouty arthritis, hyperuricemia, hypertension, cardiovascular diseases, coronary artery disease, Lesch-Nyhan syndrome, Kearns-Sayre Syndrome, nephropathy, kidney stone, renal failure, joint inflammation, arthritis, urolithiasis, lead poisoning, hyperparathyroidism, psoriasis, and sarcoidosis or hypoxanthine-guanine phosphoribosyl transferase deficiency disease.

18. The method of claim 17, wherein the disease is selected from the group consisting of gout, gout attack, gouty arthritis, and hyperuricemia.

19. A compound of 2-[4-(6-bromoquinolyl)thio]-2-ethylbutyric acid or a pharmaceutically acceptable salt thereof, Wherein:
(a) the compound has an X-ray powder diffraction (XRPD) pattern comprising peaks at diffraction angle 2θ values comprising 6.5±0.2°, 13.6±0.2°, 14.1±0.2°, 17.7±0.2°, 21.8±0.2°, 22.0±0.2°, 22.8±0.2°, 23.2±0.2°, 24.3±0.2°, 26.8±0.2°, and 27.4±0.2° when irradiated with a Cu-Kα light source (Form I);
(b) the compound has an X-ray powder diffraction (XRPD) pattern comprising peaks at diffraction angle 2θ values comprising 7.9±0.2°, 9.5±0.2°, 15.9±0.2°, 18.2±0.2°, 19.1±0.2°, 23.9±0.2°, and 26.1±0.2° when irradiated with a Cu-Kα light source (Form II); or
(c) a sodium salt of the compound has an X-ray powder diffraction (XRPD) pattern comprising peaks at diffraction angle 2θ values comprising 6.1±0.2°, 10.5±0.2°, 12.0±0.2°, 14.1±0.2°, 15.9±0.2°, 18.0±0.2°, 21.7±0.2°, 27.6±0.2°, 32.0±0.2°, 33.8±0.2°, and 36.4±0.2° when irradiated with a Cu-Kα light source (sodium salt).

20. The compound or the pharmaceutically acceptable salt thereof of claim 19, wherein the compound is in Form I, wherein relative intensity of the characteristic peaks are as follows

| Diffraction angle 2θ | Relative intensity % |
|---|---|
| 6.5 ± 0.2 | 36.1 |
| 13.6 ± 0.2 | 100.0 |
| 14.1 ± 0.2 | 14.1 |
| 17.7 ± 0.2 | 13.6 |
| 21.8 ± 0.2 | 17.0 |
| 22.0 ± 0.2 | 11.1 |
| 22.8 ± 0.2 | 16.9 |
| 23.2 ± 0.2 | 11.1 |
| 24.3 ± 0.2 | 45.6 |
| 26.8 ± 0.2 | 15.7 |
| 27.4 ± 0.2 | 36.5. |

21. The compound or the pharmaceutically acceptable salt thereof of claim 19, wherein the compound is in Form II.

22. The compound or the pharmaceutically acceptable salt thereof of claim 19, which is the sodium salt of the compound, wherein relative intensity of the characteristic peaks are as follows

| | |
|---|---|
| 27.6 ± 0.2 | 100.0 |
| 32.0 ± 0.2 | 22.0 |
| 33.8 ± 0.2 | 34.2 |
| 36.4 ± 0.2 | 22.4. |
